(19) United States
(12) Patent Application Publication
(10) Pub. No.: US 2025/0318862 A1
(43) Pub. Date: Oct. 16, 2025

(54) SYSTEM AND METHOD FOR ANCHORAGE OF LINES TO VERTEBRAE AND MINIMALLY INVASIVE SURGICAL APPLICATION

(71) Applicant: Alexander J. Butler, New York, NY (US)

(72) Inventor: Alexander J. Butler, New York, NY (US)

(21) Appl. No.: 19/176,134

(22) Filed: Apr. 10, 2025

Related U.S. Application Data
(60) Provisional application No. 63/632,329, filed on Apr. 10, 2024.
(60) Provisional application No. 63/635,400, filed on Apr. 17, 2024.

(51) Int. Cl. A61B 17/70; A61B 17/00; A61B 17/17; A61B 17/34; A61B 17/56; A61B 17/86

(52) U.S. Cl. CPC A61B 17/7053; A61B 17/00234; A61B 17/1757; A61B 17/3423; A61B 17/7067; A61B 17/86; A61B 2017/00238; A61B 2017/00477; A61B 2017/564

(57) ABSTRACT

In a system including a jig device with first and second calibrated holes, insertable through a primary incision, a cannula is configured for insertion through a secondary incision, passing through the calibrated holes. An anchor implant coupled to a line is configured for insertion through the cannula. The anchor implant includes a first tab passable through a hole in a target vertebra to anchor the implant, and a second tab disposable on the line to restrict movement of the first tab. The jig device is configured to be pulled out through the primary incision, extracting the line. The system further includes a fastener for securing the extracted line to spinal instrumentation, vertebral bone, soft tissue, or a combination thereof.

### CROSS-REFERENCE TO RELATED APPLICATIONS

The present application claims priority to U.S. Provisional Patent Application 63/632,329 filed 10 Apr. 2024, which is hereby incorporated by reference for all purposes.

### TECHNICAL FIELD

The present disclosure relates to spinal stabilization devices and methods, and more particularly to a system and method for anchorage of lines and minimally invasive surgical application.

### BACKGROUND

There is a desire to augment the stability of spinal segments either through application of only lines and dynamic materials, or in conjunction with adjacent spinal segments that have been joined together by spinal fusion. Such devices and methods may augment function, stability, position and modify displacement of spinal segments and may prevent or delay degeneration of these segments. Currently available devices applied for stabilization of the spine include screws, rods, interbody cages, disc replacement devices and off label bands or tethers. Such bands or tethers apply force to the vertebrae by wrapping around or passing through structures of the bone that are anatomic or created during surgery, without direct anchorage to the bone. Such constructs allow for motion of the lines about the structures around or through which they exert force, possibly leading to failure. Presently available devices to anchor lines to bone have not been developed for specific use in the spine, with implications for efficacy and methods of application suitable to spine surgery.

Currently employed methods for application of bands or tethers require exposure of the bones of the spine, necessitating dissection and detachment of natural soft tissues including muscle, tendon, ligament and fascia. These soft tissues are known to have a protective effect regarding stability of the spine and their damage may lead to pain, loss in function, prolonged recovery and potentially degeneration of the associated segments.

Accordingly, there is a need to augment segments of the spine by direct anchorage of lines to vertebrae, or in conjunction with those joined together by spinal fusion, with minimal dissection and detachment of these soft tissues.

### SUMMARY

In an aspect of the present disclosure, a method for anchoring a line to a target vertebra is disclosed. The method includes inserting a guide of a jig device through a primary incision made in skin of a patient. The guide has a first calibrated hole. The method includes positioning an arm of the jig device proximate to the target vertebra. The arm is coupled to the guide and has a second calibrated hole. The method includes inserting a cannula through a secondary incision made in the skin of the patient while the cannula passes through the first and second calibrated holes. The method includes inserting an anchor implant coupled to the line through the cannula. The anchor implant comprises a first tab and a second tab. The method includes passing the first tab through a hole in the target vertebra to anchor the anchor implant to the target vertebra. The method includes positioning the second tab on the line to restrict movement of the first tab. The method includes pulling the guide out through the primary incision, thereby extracting the line through the primary incision. The method includes fastening the extracted line to at least one of, a spinal instrumentation, a bone of the vertebrae, a soft tissue, or a combination thereof.

In some embodiments of the present disclosure, the method includes rotating the first tab once the first tab is through the hole in one of, a clockwise direction or an anticlockwise direction, to prevent passage back of the first tab through the hole once inserted by virtue of an oblong shape of the first tab.

In some embodiments of the present disclosure, the method includes locking the cannula to the jig device using a locking mechanism once the cannula is inserted into the first and second calibrated holes.

In some embodiments of the present disclosure, prior to inserting the anchor implant, the method includes inserting a drill bit through the cannula to create the hole in the target vertebra. The method includes inserting an awl through the cannula to create passageways in soft tissues associated with the target vertebra. The method includes inserting a trocar through the cannula to withdraw fluid from the target vertebra.

In some embodiments of the present disclosure, positioning the second tab includes deploying a clamp device on the second tab to affix the second tab to the line once advanced to a final position along the line.

In some embodiments of the present disclosure, positioning the second tab includes sliding the second tab over the line in one direction towards the first tab. The second tab comprises one of, ridges or teeth, that permits sliding in only the one direction.

In an aspect of the present disclosure, a method for anchoring a line to a target vertebra is disclosed. The method includes inserting a jig device having first and second calibrated holes through a primary incision made in skin of a patient. The method includes inserting a curved cannula through a secondary incision made in the skin of the patient such that the curved cannula passes through the first and second calibrated holes. The method includes inserting an anchor implant coupled to the line through the curved cannula. The anchor implant comprises a first tab and a second tab. The method includes passing the first tab through a hole in the target vertebra to anchor the anchor implant to the target vertebra. The method includes positioning the second tab on the line to restrict movement of the first tab. The method includes pulling the guide out through the primary incision, thereby extracting the line through the primary incision. The method includes fastening, by way of a fastener, the extracted line to at least one of, a spinal instrumentation, a bone of the vertebrae, a soft tissue, or a combination thereof.

In some embodiments of the present disclosure, the method includes rotating the first tab once the first tab is through the hole in one of, a clockwise direction or an anticlockwise direction, to prevent passage back of the first tab through the hole once inserted by virtue of an oblong shape of the first tab.

In some embodiments of the present disclosure, the method includes locking the cannula to the jig device using a locking mechanism once the cannula is inserted into the first and second calibrated holes.

In some embodiments of the present disclosure, positioning the second tab includes deploying a clamp device on the second tab to affix the second tab to the line once advanced to a final position along the line.

In some embodiments of the present disclosure, positioning the second tab includes sliding the second tab over the line in one direction towards the first tab. The second tab comprises one of, ridges or teeth, that permits sliding in only the one direction.

In an aspect of the present disclosure, a system is disclosed. The system includes a jig device comprising a guide having a first calibrated hole. The guide is configured to be inserted through a primary incision made in skin of a patient. The jig device includes an arm that is coupled to the guide, and has a second calibrated hole. The system includes a cannula configured to be inserted through a secondary incision made in the skin of the patient such that the cannula passes through the first and second calibrated holes. The system includes an anchor implant that is coupled to a line, and configured to be inserted through the cannula. The anchor implant comprises a first tab coupled to a proximal end of the line such that the first tab passes through a hole in a target vertebra to anchor the anchor implant to the target vertebra. The anchor implant comprises a second tab that is disposed onto the line, and configured to restrict a movement of the first tab. Once the anchor implant is anchored to the target vertebra, the guide is configured to be pulled out through the primary incision thereby extracting the line through the primary incision.

In some embodiments of the present disclosure, the guide has a first end and a second end such that the first end of the guide comprises the first calibrated hole.

In some embodiments of the present disclosure, the arm is an L-shaped structure having a first end and a second end such that first end of the arm comprises the second calibrated hole and the second end of the arm is coupled to the second end of the guide.

In some embodiments of the present disclosure, the first tab has an oblong shape such that when the first tab is passed through the hole of the target vertebra, the first tab is rotated by way of the line in one of, a clockwise direction or an anticlockwise direction, to prevent passage back of the first tab through the hole once inserted by virtue of the oblong shape.

In some embodiments of the present disclosure, the system includes a locking mechanism configured to lock the cannula to the jig device once the cannula is inserted into the first and second calibrated holes.

In some embodiments of the present disclosure, the system includes a drill bit. Prior to insertion of the anchor implant into the cannula, the drill bit is inserted through the cannula to create a hole in the target vertebra.

In some embodiments of the present disclosure, the system includes an awl. Prior to insertion of the anchor implant into the cannula, the awl is inserted through the cannula to create passageways in soft tissues associated with the bone of the vertebrae.

In some embodiments of the present disclosure, the system includes a trocar. Prior to insertion of the anchor implant into the cannula, the trocar is inserted through the cannula to withdraw fluid from the target vertebrae.

In some embodiments of the present disclosure, the system includes a fastener configured to fasten the line that is extracted from the primary incision with one of, one or more spinal instrumentations, a bone of the vertebrae, a soft tissue, or a combination thereof.

In some embodiments of the present disclosure, the second tab comprises a deployable clamp device that affixes the second tab to the line once advanced to a final position along the line.

In some embodiments of the present disclosure, the second tab comprises one of, ridges or teeth, that permits sliding the second tab over the line in one direction. The one direction is towards the first tab.

In an aspect of the present disclosure, a system is disclosed. The system includes a jig device that is configured to be inserted through a primary incision made in skin of a patient. The jig device comprises a first calibrated hole and a second calibrated hole. The system includes a curved cannula configured to be inserted through a secondary incision made in the skin of the patient such that the cannula passes through the first and second calibrated holes. The system includes an anchor implant that is attached to a line, and configured to be inserted through the curved cannula. The anchor implant comprises a first tab attached to a proximal end of the line such that the first tab passes through a hole of a bone of the vertebrae to anchor the anchor implant to the bone. The anchor implant comprises a second tab that is disposed on the line, and configured to restrict a movement of the first tab. Once the anchor implant is anchored to the bone, the guide is configured to be pulled out through the primary incision thereby extracting the line through the primary incision.

In some embodiments of the present disclosure, the jig device has a L-shaped structure. The jig device comprises a first end and a second end such that the first end of the jig device comprises the first calibrated hole and the second end of the jig device comprises the second calibrated hole.

In some embodiments of the present disclosure, the first tab has an oblong shape. When the first tab is passed through the hole of the target vertebra, the first tab is rotated in one of, a clockwise direction or an anticlockwise direction, to prevent passage back through the hole by virtue of the oblong shape.

In some embodiments, the first tab comprises a first end and a second end and is configured to fold with application of a force to each of the first end and the second end such that the first tab may be inserted through the hole of the target vertebra in a folded position. Upon passage of the folded first tab through the hole and removal of the forces on the first end and the second end, the tab is configured to unfold. In an unfolded position, the first tab is prevented from passing back through the hole. In some embodiments, the first tab includes a spring positioned between the first end and the second end, such that upon application of a force to the first end and the second end a straightening force of the spring is overcome resulting in the spring being bent or flexed such that the first end and the second end are moved to a position adjacent to each other. In certain embodiments, the spring comprises a flat spring, a wound wire spring, a torsion spring, a compression spring, or any other type of spring. In some embodiments, the spring may be comprises of metal, plastic, or some other biocompatible material

In some embodiments, the first tab, the second tab, or the spring may include one or more antibacterial or antibiotic compounds.

In some embodiments of the present disclosure, the system includes a locking mechanism configured to lock the cannula to the jig device once the cannula is inserted into the first and second calibrated holes.

In some embodiments of the present disclosure, the system includes a curved and flexible drill bit. Prior to insertion of the anchor implant into the curved cannula, the curved and flexible drill bit drill bit is inserted through the curved cannula to create a hole in the bone of the vertebrae.

In some embodiments of the present disclosure, the system includes a curved and flexible awl. Prior to insertion of the anchor implant into the curved cannula, the curved and flexible awl is inserted through the curved cannula to create passageways in soft tissues associated with the bone of the vertebrae.

In some embodiments of the present disclosure, the system includes a curved and flexible trocar. Prior to insertion of the anchor implant into the curved cannula, the curved and flexible trocar is inserted through the curved cannula to withdraw fluid from the target vertebrae.

In an aspect of the present disclosure, a system is disclosed. The system includes a jig device that is configured to be inserted through a primary incision made in skin of a patient. The jig device comprises a first calibrated hole and a second calibrated hole. The system includes a cannula configured to be inserted through a secondary incision made in the skin of the patient such that the cannula passes through the first and second calibrated holes. The system includes an anchor implant that is attached to a line, and configured to be inserted through the curved cannula. The anchor implant comprising a plurality of threads such that the anchor implant is screwed inside the hole. Once the anchor implant is screwed inside the hole of the bone, the jig device is pulled out through the primary incision thereby extracting the line through the primary incision.

In some embodiments of the present disclosure, the jig device has a L-shaped structure. The jig device comprises a first end and a second end such that the first end of the jig device comprises the first calibrated hole and the second end of the jig device comprises the second calibrated hole.

In some embodiments of the present disclosure, the jig device comprises a guide having a first calibrated hole. The guide is configured to be inserted through the primary incision made in the skin of the patient. The jig device comprises an arm that is coupled to the guide, and has the second calibrated hole.

In some embodiments of the present disclosure, the guide is pulled out through the primary incision thereby extracting the line through the primary incision.

In some embodiments of the present disclosure, the cannula is a straight cannula.

In some embodiments of the present disclosure, the cannula is a curved cannula.

In some embodiments of the present disclosure, the first tab has an oblong shape. When the first tab is passed through the hole of the target vertebra, the first tab is rotated in one of, a clockwise direction or an anticlockwise direction, to prevent passage back through the hole by virtue of the oblong shape.

In some embodiments of the present disclosure, the system includes a locking mechanism configured to lock the cannula to the jig device once the cannula is inserted into the first and second calibrated holes.

In some embodiments of the present disclosure, the system includes a drill bit. Prior to insertion of the anchor implant into the cannula, the curved and flexible drill bit is inserted through the cannula to create a hole in the bone of the vertebrae. The system includes an awl. Prior to insertion of the anchor implant into the cannula, the awl is inserted through the cannula to create passageways in soft tissues associated with the bone of the vertebrae. The system includes a trocar. Prior to insertion of the anchor implant into the cannula, the trocar is inserted through the cannula to withdraw fluid from the target vertebrae.

In an aspect of the present disclosure, an anchor implant for a vertebra is disclosed. The anchor implant includes a first tab attached to a proximal end of a line such that the first tab passes through a hole of a bone of a target vertebra to anchor the anchor implant to the bone. The first tab has an oblong shape such that when the first tab is passed through the hole of the bone, the first tab is rotated in one of, a clockwise direction or an anticlockwise direction, to prevent passage back through the hole by virtue of the oblong shape of the first tab. The anchor implant includes a second tab that is disposed on the line, and configured to restrict a movement of the first tab. To restrict the movement of the first tab, the second tab is pushed towards the first tab such that the first tab and the second tab are held tightly on either side of the bone of the target vertebrae.

In some embodiments of the present disclosure, the first tab and the second tab attached to the line are passed through a cannula coupled to a jig device through first and second calibrated holes of the jig device such that the jig device is positioned proximate to a target vertebra.

In an aspect of the present disclosure, a system is disclosed. The system includes a jig device configured to be inserted through a primary incision made in skin of a patient. The jig device comprises first and second calibrated holes. The system includes a cannula configured to be inserted through a secondary incision made in the skin of the patient such that the cannula passes through the first and second calibrated holes. The system includes an anchor implant that is coupled to a line, and configured to be inserted through the cannula. The anchor implant is configured to be anchored to a hole in the target vertebra. The jig device configured to be pulled out through the primary incision when the anchor implant is anchored to the target vertebra thereby extracting the line through the primary incision. The line is fastened to one of, one or more spinal instrumentations, a bone of the vertebrae, or a combination thereof.

In some embodiments of the present disclosure, the jig device comprises a guide having a first end and a second end such that the first end of the guide comprises the first calibrated hole. The jig device comprises an arm having a first end and a second end such that first end of the arm comprises the second calibrated hole and the second end of the arm is coupled to the second end of the guide.

In some embodiments of the present disclosure, when the jig device has the guide and the arm, the cannula is a straight cannula.

In some embodiments of the present disclosure, the cannula is a curved and flexible cannula.

In some embodiments of the present disclosure, the anchor implant comprises a first tab coupled to a proximal end of the line such that the first tab passes through the hole in the target vertebra to anchor the anchor implant to the target vertebra. The first tab has an oblong shape such that when the first tab is passed through the hole of the target vertebra, the first tab is rotated by way of the line in one of, a clockwise direction or an anticlockwise direction, to prevent passage back of the first tab through the hole once inserted by virtue of the oblong shape. The anchor implant comprises a second tab that is disposed onto the line, and configured to restrict a movement of the first tab.

In some embodiments of the present disclosure, the anchor implant comprises a first tab having a plurality of threads such that when the first tab of the anchor implant is received at the hole through the cannula, the first tab is screwed inside the hole to restrict the movement of the first tab.

In some embodiments of the present disclosure, the system includes a locking mechanism configured to lock the cannula to the jig device once the cannula is inserted into the first and second calibrated holes.

In some embodiments of the present disclosure, the system includes a drill bit. Prior to insertion of the anchor implant into the cannula, the drill bit is inserted through the cannula to create a hole in the target vertebra. The system includes an awl. Prior to insertion of the anchor implant into the cannula, the awl is inserted through the cannula to create passageways in soft tissues associated with the bone of the vertebrae. The system includes trocar. Prior to insertion of the anchor implant into the cannula, the trocar is inserted through the cannula to withdraw fluid from the target vertebrae.

In some embodiments of the present disclosure, the second tab comprises a deployable clamp device that affixes the second tab to the line once advanced to a final position along the line.

In some embodiments of the present disclosure, the second tab comprises one of, ridges or teeth, that permits sliding the second tab over the line in one direction. The one direction is towards the first tab.

The foregoing general description of the illustrative embodiments and the following detailed description thereof are merely exemplary embodiments of the teachings of this disclosure and are not restrictive.

### BRIEF DESCRIPTION OF THE DRAWINGS

The following detailed description of the preferred embodiments of the present disclosure will be better understood when read in conjunction with the appended drawings. The present disclosure is illustrated by way of example, and not limited by the accompanying figures, in which like references indicate similar elements.

FIG. 1A illustrates a system installed on a coronal view of a vertebral column of a patient for anchorage of one or more lines to a target vertebra of the vertebral column, according to an embodiment of the present disclosure;

FIG. 1B illustrates the system installed on a sagittal view of the vertebral column of the patient for anchorage of the line to the target vertebra of the vertebral column, according to an embodiment of the present disclosure;

FIG. 1C illustrates another system installed on an axial view of the target vertebra of the patient for anchorage of the line to the target vertebra, according to an embodiment of the present disclosure

FIG. 2 illustrates a drill bit, a trocar, and an applicator device of the system of FIG. 1C, according to embodiments of the present disclosure;

FIG. 3A illustrates an axial view of the target vertebra where the anchor implant is installed onto a spinous process of the target vertebra, according to an embodiment of the present disclosure;

FIG. 3B illustrates another axial view of the target vertebra where the anchor implant is installed onto the spinous process of the target vertebra, according to an embodiment of the present disclosure;

FIG. 3C illustrates another axial view of the target vertebra where the anchor implant is installed onto the spinous process, according to an embodiment of the present disclosure;

FIG. 3D illustrates another axial view of the target vertebra where the anchor implant is installed onto a bone of the target vertebra, according to an embodiment of the present disclosure;

FIG. 3E illustrates a profile view of an embodiment of an anchor implant in a deployed state, according to an embodiment of the present disclosure;

FIG. 3F illustrates a profile view of an embodiment of an anchor implant in a pre-deployed state, according to an embodiment of the present disclosure;

FIGS. 4A-4C illustrate various views of the target vertebra showing holes and tunnels created for anchoring the anchor implant and the line, according to embodiments of the present disclosure;

FIG. 5 illustrates the system of FIG. 1A installed on a coronal view of the vertebral column, according to embodiments of the present disclosure;

FIG. 6 illustrates a posterior view of attachment of the lines with the vertebral column of the patient, according to embodiment of the present disclosure; and

FIG. 7 illustrates a flowchart of a method for anchoring the line to the target vertebra, according to embodiments of the present disclosure.

### DETAILED DESCRIPTION

The detailed description of the appended drawings is intended as a description of the currently preferred embodiments of the present disclosure, and is not intended to represent the only form in which the present disclosure may be practiced. It is to be understood that the same or equivalent functions may be accomplished by different embodiments that are intended to be encompassed within the spirit and scope of the present disclosure.

The present disclosure relates to systems and methods for minimally invasive spinal stabilization through the anchorage of lines to vertebrae. This approach allows for augmentation of spinal segments with minimal dissection or removal of overlying soft tissue, addressing a significant need in spine surgery. The system comprises a specialized jig device designed to access vertebrae through small incisions. This jig device works in conjunction with a cannula, enabling the precise delivery of surgical instruments and implants to targeted vertebral sites while navigating around soft tissues. The design allows for both percutaneous and minimally invasive open surgical approaches, providing flexibility to surgeons based on patient needs and surgical goals. Central to the system is an anchor implant that can be delivered through the cannula and secured within a hole or tunnel created in the vertebral bone. The anchor implant's design allows it to be inserted in a low-profile configuration and then deployed to prevent pull-out, providing a stable attachment point for tethering lines.

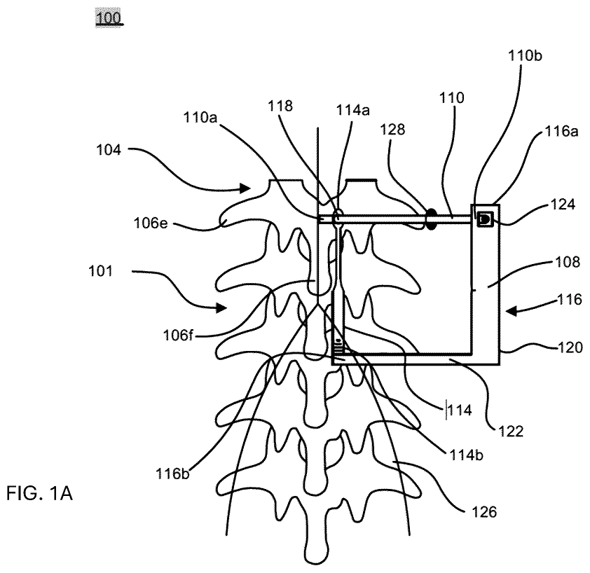

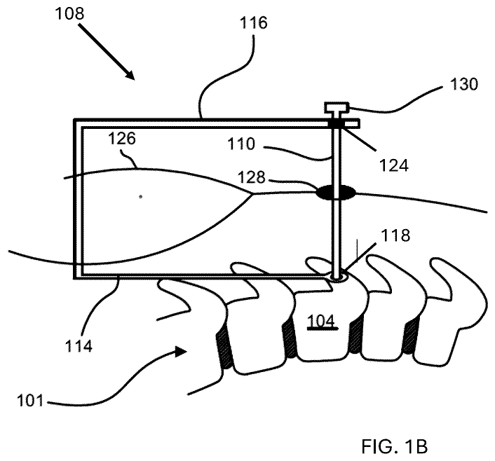

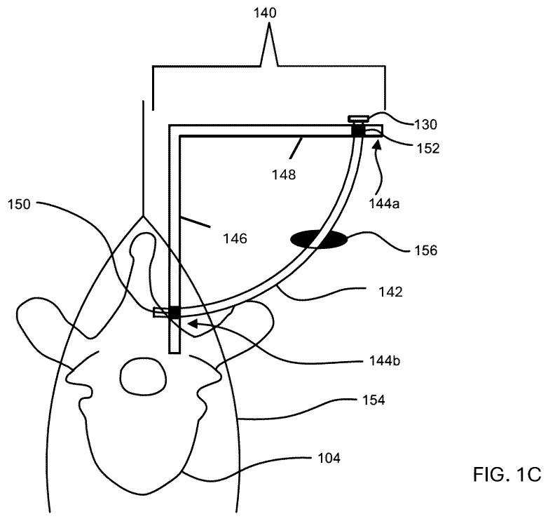

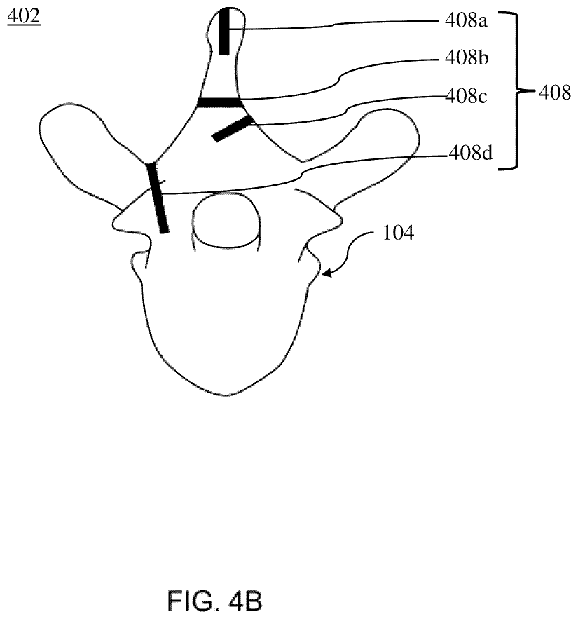

FIG. 1A illustrates a system 100 installed on a coronal view of a vertebral column 101 of a patient for anchorage of one or more lines 102 (shown later in FIGS. 4A-6) to a target vertebra 104 of the vertebral column 101, according to an embodiment of the present disclosure. According to embodiments of the present disclosure, the system 100 may be utilized to anchor the one or more lines 102 (hereinafter individually referred to and designated as “the line 102” and collectively referred to and designated as “the lines 102”) to the target vertebra 104 of the vertebral column 101 of the patient in minimally invasive surgical applications. Specifically, the system 100 may be utilized to anchor the line 102 (hereinafter interchangeably referred to and designated as “the tether 102”) to the target vertebra 104 of the vertebral column 101 of the patient to help correct and control various conditions affecting the vertebral column 101 (hereinafter interchangeably referred to as “the spine”). As illustrated, the vertebral column 101 or the spine, is made up of a series of bones called vertebrae, which are stacked one above the other to form the backbone. Each typical vertebra may have several parts that contribute to its structure and function. As shown, a vertebral body is the thick, anterior (front) part of the vertebra that bears weight. Each vertebra (e.g., the target vertebra 104) may be formed by two pedicles and two laminae. The lamina is the flat, bony plate that forms the posterior (back) part of the arch, connecting a transverse process 106e to a spinous process 106f. Specifically, the transverse process 106e are bony projections that extend laterally (sideways) from either side of the vertebra and serve as attachment points for muscles and ligaments. The spinous process 106f projects backward from the midline of the vertebra and can be felt as the bony bumps along the back. In some embodiments of the present disclosure, the line 102 may anchor or tether the bones of the target vertebra 104 such that the line couples to the vertebrae, and may pass through anatomic passageways in the vertebrae or may pass through holes in the bone created by a drill bit, awl or other instrument. Alternatively, the line may loop or wrap around anatomic structures of the vertebrae. The line 102 may be incorporated by construction with or used as part of the system 100 in conjunction with the anchor implant 112 to prevent motion, toggle, slippage or translation about the vertebrae it serves to tether or anchor. In some embodiments of the present disclosure, the line 102 may be, but is not limited to, a flexible cord, a flexible cable, a flexible rope, or the like. Embodiments of the present disclosure are intended to include and/or otherwise cover any type of the line/tether 102, known to a person having ordinary skill in the art, without deviating from the scope of the present disclosure.

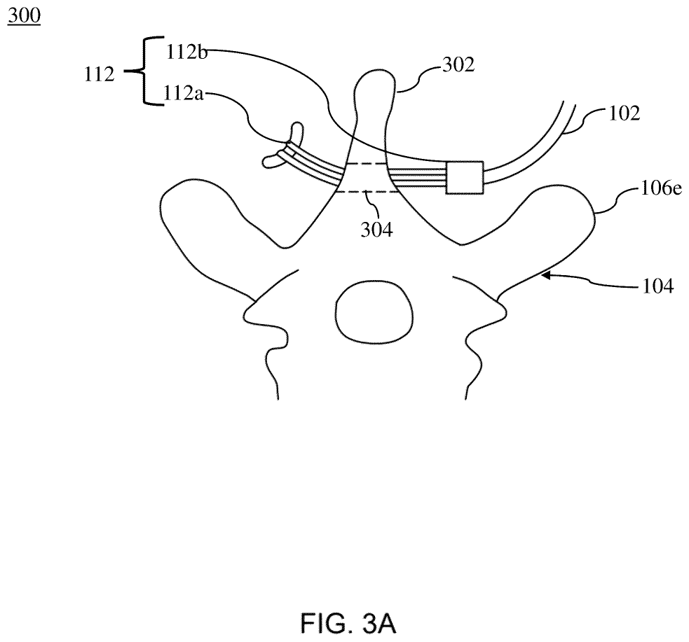

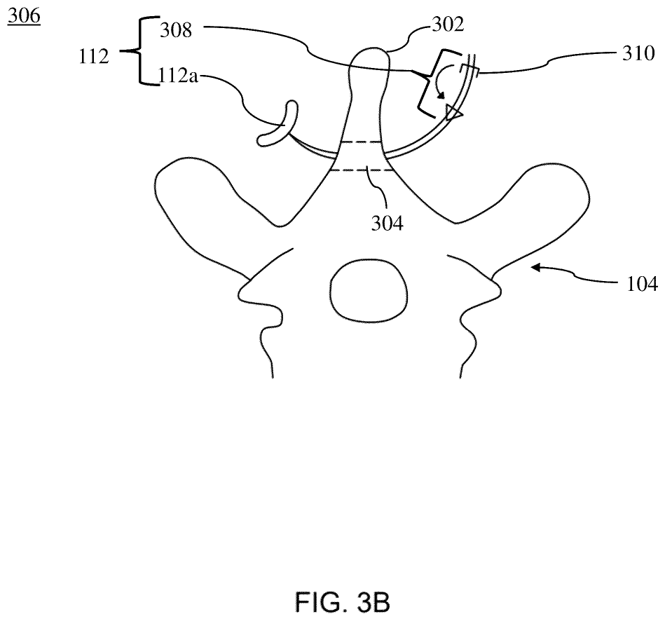

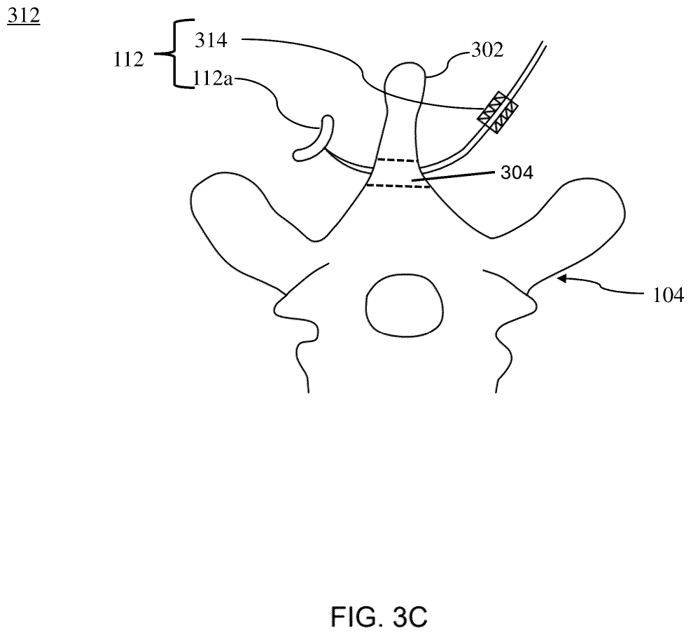

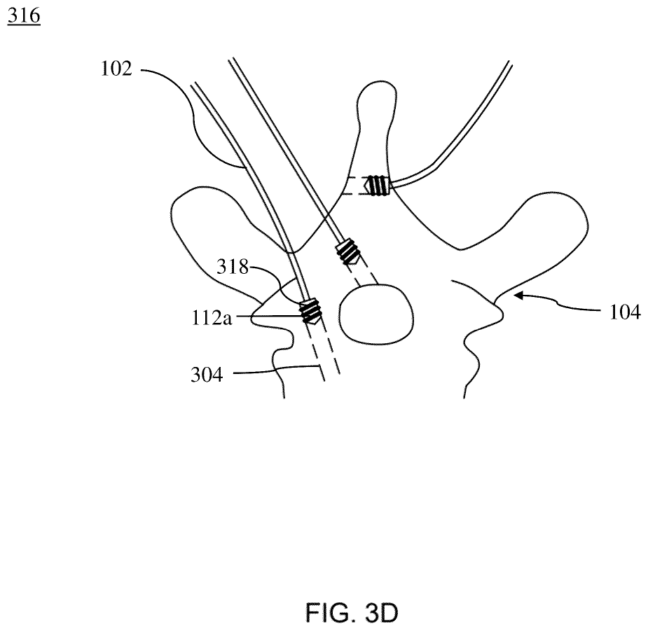

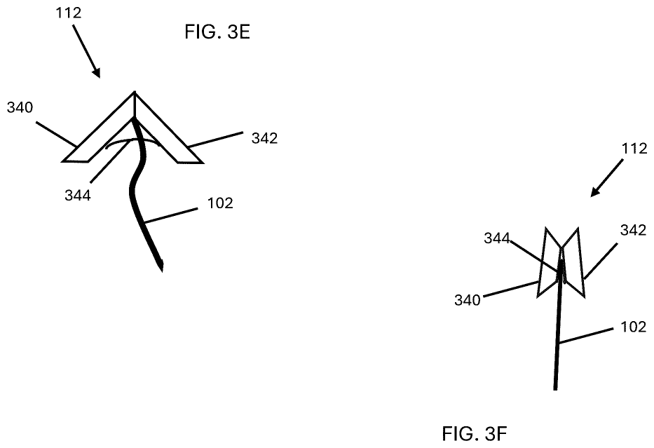

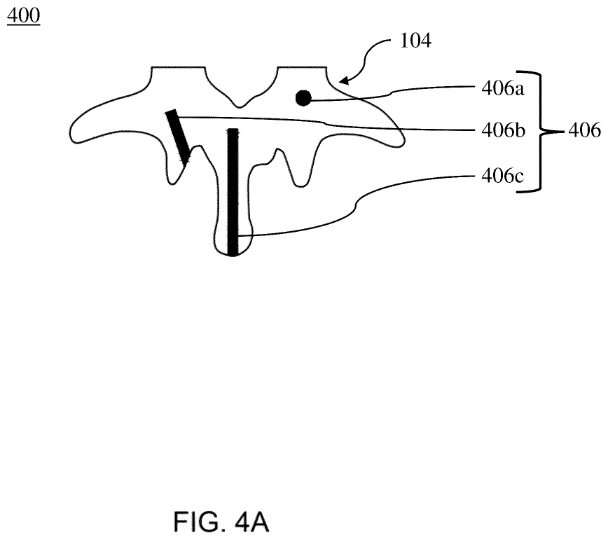

The system 100 may include a jig device 108, a cannula 110, and an anchor implant 112 (shown later in FIGS. 3A-3D). Specifically, the jig device 108, the cannula 110, and the anchor implant 112 may be assembled to access the target vertebra 104 of the vertebral column 101 of the patient in minimally invasive surgical applications. The jig device 108 may be utilized to affix the anchor implant 112 and/or the line/tether 102 to the target vertebra 104 of the vertebral column 101 of the patient or associated soft tissues.

In some embodiments of the present disclosure, the jig device 108 may include a guide 114 and an arm 116 such that the guide 114 and the arm 116 are coupled to each other to form the jig device 108. In some embodiments of the present disclosure, the guide 114 may be configured to be laid directly on the targeted vertebra or soft tissue to access the targeted vertebra or soft tissue. In some other embodiments of the present disclosure, the guide 114 may be configured to be passed beneath overlying soft tissues to access the targeted vertebra or soft tissue percutaneously without direct surgical exposure. Further, the guide 114 may have a first end 114a and a second end 114b such that the first end 114a of the guide 114 has a first calibrated hole 118. In some embodiments of the present disclosure, the arm 116 may have a first part 120 and a second part 122 forming a L-shaped structure. Further, the arm 116 may have a first end 116a and a second end 116b such that the first end 116a of the arm 116 has a second calibrated hole 124. As illustrated, the second calibrated hole 124 is provided on the first end 116a of the arm 116 (i.e., the first part 120 of the arm 116). In some embodiments of the present disclosure, the second part 122 may be utilized as a handle to manipulate the jig device 108 and to position the jig device 108 at an appropriate location over the vertebral column 101. Further, the first calibrated hole 118 provided in the guide 114 and the second calibrated hole 124 provided in the arm 116 (i.e., the first part 120 of the arm 116) are configured to allow for reception of the cannula 110 and/or any other such instruments to access the target vertebrae or associated soft tissues in the vertebral column 101. Specifically, the cannula 110 and/or any other such instruments may pass through overlying soft tissues to access the guide 114 overlying the spine or associated soft tissue when used percutaneously.

The second end 114b of the guide 114 and the second end 116b of the arm 116 may be coupled to form the jig device 108. In some embodiments of the present disclosure, the second end 114b of the guide 114 and the second end 116b of the arm 116 may be removably coupled to each other to form the jig device 108. In some other embodiments of the present disclosure, the second end 114b of the guide 114 and the second end 116b of the arm 116 may be fixedly coupled to each other to form the jig device 108.

In some embodiments of the present disclosure, the guide 114 and the arm 116 may be made up of a material such as, but not limited to, autograft, allograft, polyether ether ketone (PEEK), polyethylene, titanium, stainless steel, or the like. Embodiments of the present disclosure are intended to include and/or otherwise cover any type of the biocompatible material that is rigid and/or malleable in structure, known to a person having ordinary skill in the art, without deviating from the scope of the present disclosure.

As discussed, the arm 116 may have a L-shaped structure. In some embodiments of the present disclosure, the arm 116 may have a hollow structure. In some other embodiments of the present disclosure, the arm 116 may have a solid structure. Similarly, in some embodiments of the present disclosure, the guide 114 may have hollow structure. In some other embodiments of the present disclosure, the guide 114 may have a solid structure. Although FIG. 1A illustrates that the arm 116 has the L-shaped structure, it will be apparent to a person skilled in the art that the scope of the present disclosure is not limited to it. In various other embodiments of the present disclosure, the arm 116 may have any suitable shape that facilitates efficient implementation of the system 100 for anchorage of the line 102 to the target vertebra 104 of the vertebral column 101 of the patient, without deviating from the scope of the present disclosure. As illustrated, the guide 114 may be configured to be inserted through a primary incision 126 made in skin of the patient. In some embodiments of the present disclosure, the primary incision 126 may enable insertion of the guide 114 beneath the overlying soft tissues of the vertebral column 101 of the patient to access various portions of the vertebral column 101.

The cannula 110 may be a hollow tubular structure configured to be inserted through a secondary incision 128 made in the skin of the patient. The cannula 110 may be inserted through the secondary incision 128 such that the cannula 110 passes through the first calibrated hole 118 and the second calibrated hole 124. In some embodiments of the present disclosure, the cannula 110 may have a proximal end 110a and a distal end 110b such that once the cannula 110 is secured in the first calibrated hole 118 and the second calibrated hole 124 of the jig device 108, the anchor implant 112 is inserted by way of an applicator device (not shown) from the distal end 110b through the cannula 110 and is received proximate to the target vertebra 104 of the vertebral column 101 of the patient via the proximal end 110a. In other words, once the cannula 110 is passed through the first calibrated hole 118 and the second calibrated hole 124 of the jig device 108 via the secondary incision 128, the anchor implant 112 is passed through the cannula 110 to access the target vertebra 104 of the vertebral column 101 of the patient. As illustrated in FIG. 1A, the cannula 110 is used through a formal surgical exposure. Specifically, due to the use of the cannula 110 through the formal surgical exposure, the cannula 110 is a straight cannula that allows direct access to the guide 114 of the jig device 108. In some embodiments of the present disclosure, the cannula 110 may have a flexible, curved, and/or semi-rigid elongated body having a proximal end and a distal end. Embodiments of the present disclosure are intended to include and/or otherwise cover any type of the cannula 110, known to a person having ordinary skill in the art, without deviating from the scope of the present disclosure.

As shown in FIG. 3B, in some embodiments of the present disclosure, the system 100 may further include a locking mechanism 130. The locking mechanism 130 may be configured to lock and/or secure the cannula 110 to the jig device 108 once the cannula 110 is passed through the first calibrated hole 118 and the second calibrated hole 124 of the jig device 108. In some embodiments of the present disclosure, the locking mechanism 130 may include, but is not limited to, a threaded locking mechanism, a standard or modified Luer lock connection, a snap-fit mechanism, a rotational lock, a slide-lock mechanism, a magnetic locking mechanism, and the like. Embodiments of the present disclosure are intended to include and/or otherwise cover any type of the locking mechanism 130 that may facilitate in securing and/or locking the cannula 110 to the jig device 108 once the cannula 110 is passed through the first calibrated hole 118 and the second calibrated hole 124 of the jig device 108 via the secondary incision 128, known to a person having ordinary skill in the art, without deviating from the scope of the present disclosure.

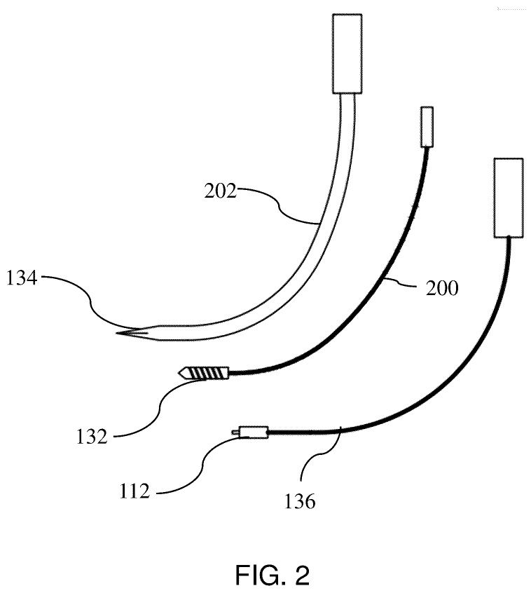

The system 100 may further include a drill bit 132, an awl (not shown), a trocar 134, and an applicator device 136 (each as shown later in FIG. 2). In some embodiments of the present disclosure, prior to insertion of the anchor implant 112 into the cannula 110, the drill bit 132 may be inserted through the cannula 110 to create a hole in the target vertebra 104. Similarly, to create passageways in soft tissues, the awl may be inserted through the cannula 110 such that the awl can be operated to create passageways in soft tissues associated with the bone of the target vertebrae 104 prior to insertion of the anchor implant 112 through the cannula 110. Similarly, to withdraw fluid from the target vertebrae, the trocar 134 may be inserted through the cannula 110 such that the trocar 134 is operated to withdraw fluid from the target vertebrae 104 prior to insertion of the anchor implant 112 into the cannula 110. Once the passageway in the soft tissues and the hole in the bone of the target vertebra 104 is created, the fluid may be withdrawn from the target vertebrae and the anchor implant 112 attached to the line 102 may be inserted into the cannula 110 by way the applicator device 136.

In some embodiments of the present disclosure, the anchor implant 112 may include, but is not limited to, suture, barbs, threads, ridges, claws, hooks, tines, knots, and the like. Embodiments of the present disclosure are intended to include and/or otherwise cover any type of the anchor implant 112 to provide anchorage against and/or within the target vertebra 104 or associated soft tissue, known to a person having ordinary skill in the art, without deviating from the scope pf the present disclosure. In some embodiments of the present disclosure, the anchor implant 112 may be made from materials including, but not limited to, autograft, allograft, polyether ether ketone (PEEK), polyethylene, titanium, stainless steel or other biocompatible substance and be rigid or malleable in structure to facilitate tethering or anchorage to the vertebrae. Embodiments of the present disclosure are intended to include and/or otherwise cover any type of the material for the anchor implant 112, known to a person having ordinary skill in the art, without deviating from the scope of the present disclosure. In some embodiments of the present disclosure, the anchor implant 112 may be incorporated with the line 102 in prefabricated construction. In some other embodiments of the present disclosure, the anchor implant 112 and the line 102 may be applied separately to achieve fixation, or to prevent motion, toggle, slippage or translation on the target vertebra 104.

In operation, a medical practitioner begins by making the primary incision 126 and the secondary incision 128 in the skin of the patient. Further, the guide 114 of the jig device 108 is then carefully passed through the primary incision 126, navigating beneath the soft tissues overlying the vertebral column 101 to access various portions of the target vertebrae 104. In some embodiments of the present disclosure, to ensure accurate localization, the placement of the jig device 108 may be guided by one or more techniques such as, but not limited to, intraoperative X-ray, fluoroscopy, three-dimensional spinal navigation technique, augmented and mixed reality technique, and the like. Embodiments of the present disclosure are intended to include and/or otherwise cover any type of the technique, known to a person having ordinary skill in the art, without deviating from the scope of the present disclosure. Specifically, the one or more techniques continue to play a crucial role throughout the procedure, confirming anatomic targets and providing radiographic visualization of the devices and spinal bones to the medical practitioner, as the soft tissue overlying target spinal segments remains intact. In an exemplary scenario, when the system 100 is used through a formal surgical exposure, the cannula 110 may be passed through the second calibrated hole 124 provided in the arm 116 of the jig device 108 and guided through the overlying soft tissue via the secondary incision 128. The cannula 110 is advanced until the cannula 110 accesses the first calibrated hole 118 provided in the guide 114 of the jig device 108. Further, the locking mechanism 130 may be engaged to secure the cannula 110 in place, affixed to the jig device 108. Further, the drill bit 132 is then introduced through the cannula 110 to access the target vertebra 104. The drill bit 132 may be introduced through the cannula 110 to access the target vertebra 104 to create a hole and/or passageway in a bone of the target vertebra 104. In some cases, the awl and the trocar 134 may be introduced through the cannula 110 to access the target vertebra 104 to create passageways in soft tissues associated with the bone of the target vertebrae 104 and to withdraw fluid from the target vertebrae 104, respectively.

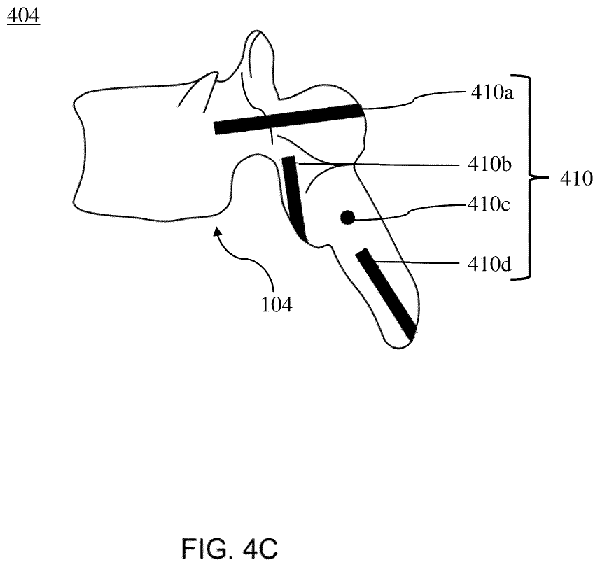

Following the creation of the hole (hereinafter interchangeably referred to and designated as “the tunnel”) in the bone of the target vertebrae 104, the applicator device 136 may be employed to deliver the anchor implant 112 through the cannula 110 to the hole in the bone of the target vertebra 104. Specifically, the applicator device 136 may be used to pass the anchor implant 112 attached to the line 102 through the second calibrated hole 124 in the guide 114 of the jig device 108 and into the hole created in the bone of the target vertebra 104. Once the anchor implant 112 is anchored to the bone of the target vertebra 104, the line 102 that extends beyond the anchor implant 112 through the first calibrated hole 118 is carefully pulled by way of the guide 114 such that the line 102 is extracted through the primary incision 126 while the guide 114 is removed from the primary incision 126. In other words, when the anchor implant 112 is anchored to the bone of the target vertebra 104, the guide 114 may be pulled through the primary incision 126, this action pulls the line 102 engaged with the guide 114 through the secondary incision 128 and overlying soft tissue, bringing the line 102 out through the primary incision 126. In some embodiments of the present disclosure, the line 102 may be fastened using a bone anchor mechanism, to one of, but not limited to, adjacent vertebrae, to a previously placed spinal instrumentation (such as a plurality of spinal instrumentation 500 as shown in FIG. 5) via a clamp, a knot mechanism, to other areas of the spine, and the like to complete the minimally invasive tethering procedure. Embodiments of the present disclosure are intended to include and/or otherwise cover any type of the bone anchor mechanism to fasten the line 102 to any areas of the spine, known to a person having ordinary skill in the art, without deviating from the scope of the present disclosure.

FIG. 1B illustrates the system 100 installed on a sagittal view of the vertebral column 101 of the patient for anchorage of the line 102 to the target vertebra 104 of the vertebral column 101, according to an embodiment of the present disclosure. As illustrated, the system 100 of FIG. 1B is implemented in a manner similar to that of FIG. 1A, however, the placement of the primary incision 126 and the secondary incision 128 is different when compared to FIG. 1A. In this configuration, as illustrated, the primary incision 126 and the secondary incision 128 are colinear, meaning they are aligned along the same line or axis. This colinear arrangement of the primary incision 126 and the secondary incision 128 provides an alternative approach for minimally invasive access to the target vertebra 104. The colinear placement of the primary incision 126 and the secondary incision 128 may offer advantages in specific surgical scenarios such as, but not limited to, simplifying the surgical approach by allowing the medical practitioner to work along a single line of access. This configuration might be particularly beneficial in cases where anatomical constraints or patient-specific factors make a single-line approach more favorable. The jig device 108, including the guide 114 and the arm 116, is positioned in a manner similar to that described in FIG. 1A. However, the alignment of the arm 116 may be adjusted to accommodate the colinear incision placement. The procedure for anchoring the line 102 to the target vertebra 104 follows the same steps as described for FIG. 1A, including the use of the drill bit, awl, trocar, and applicator device as needed. The colinear incision arrangement does not fundamentally alter the function of the system components or the overall surgical technique, but rather provides an alternative approach that may be preferred in certain clinical situations. This colinear configuration demonstrates the versatility of the system 100, allowing it to be adapted to various surgical approaches while maintaining its core functionality for minimally invasive vertebral anchoring.

FIG. 1C illustrates another system 138 installed on an axial view of the target vertebra 104 of the patient for anchorage of the line 102 to the target vertebra 104, according to an embodiment of the present disclosure. As illustrated, the system 138 may include a jig device 140, a cannula 142, and the anchor implant 112 (shown later in FIGS. 3A-3D). Specifically, the jig device 140 and the cannula 142 are substantially similar to the jig device 108 and the cannula 110 of FIG. 1A. However, the jig device 140 of FIG. 1C does not include the guide 114. Further, the arm 144 may have a first end 144a and a second end 144b such that the second end 144b of the arm 144 has a first calibrated hole 150 and the first end 144a of the arm 144 has a second calibrated hole 152. In other embodiments, the jig device 140 may have the first end 144a and the second end 144b such that the first end 144a of the jig device 144 has the first calibrated hole 150 and the second end 144b of the jig device 144 has the second calibrated hole 152. As illustrated, the first calibrated hole 150 is provided on the second end 144b of the arm 144 (i.e., a first part 146 of the arm 144) and the second calibrated hole 152 is provided on the first end 144a of the arm 144 (i.e., the second part 148 of the arm 144). In some embodiments of the present disclosure, the second part 148 may be utilized as a handle to manipulate the jig device 140 and to position the jig device 140 at appropriate location over the vertebral column 101. Further, the first calibrated hole 150 and the second calibrated hole 152 of the jig device 140 are configured to allow for reception of the cannula 142 and/or any other such instruments to access the target vertebrae or associated soft tissues in the vertebral column 101. Specifically, the cannula 142 and/or any other such instruments may pass through overlying soft tissues to access the second calibrated hole 152 of the jig device 140 inserted through a primary incision 154 made on the skin of the patient such that the jig device 140 overly the vertebral column 101 or associated soft tissue when used percutaneously. Further, the cannula 142 is a flexible and curved cannula that may be adapted to permit orthogonal passage through the first calibrated hole 150 and the second calibrated hole 152 of the jig device 140 via a secondary incision 156 made on the skin of the patient, relatively, having passed through overlying soft tissues when used percutaneously. Further, the locking mechanism 130 may be configured to lock and/or secure the cannula 142 to the jig device 140 once the cannula 142 is passed through the first calibrated hole 150 and the second calibrated hole 152 of the jig device 140.

In operation, a medical practitioner begins by making the primary incision 154 and the secondary incision 156 in the skin of the patient. Further, the jig device 140 is then carefully passed through the primary incision 154, navigating beneath the soft tissues overlying the vertebral column 101 to access various portions of the target vertebrae 104. In some embodiments of the present disclosure, to ensure accurate localization, the placement of the jig device 140 may be guided by one or more techniques such as, but not limited to, intraoperative X-ray, fluoroscopy, three-dimensional spinal navigation technique, augmented and mixed reality technique, and the like. Embodiments of the present disclosure are intended to include and/or otherwise cover any type of the technique, known to a person having ordinary skill in the art, without deviating from the scope of the present disclosure. Specifically, the one or more techniques continue to play a crucial role throughout the procedure, confirming anatomic targets and providing radiographic visualization of the devices and spinal bones to the medical practitioner, as the soft tissue overlying target spinal segments remains intact. In an exemplary scenario, when the system 140 is used percutaneously, the cannula 142 (i.e., the curved and flexible cannula) may be passed through the second calibrated hole 152 provided in the second part 148 of the arm 144 of the jig device 140 and guided through the overlying soft tissue via the secondary incision 156. The cannula 142 is advanced until the cannula 142 accesses the first calibrated hole 150 provided in the first part 146 of the arm 144 of the jig device 140. Further, the locking mechanism 130 may be engaged to secure the cannula 142 in place, affixed to the jig device 140. In some embodiments of the present disclosure, the drill bit 132 may be then introduced through the cannula 142 to access the target vertebra 104. Specifically, the drill bit 132 may be introduced through the cannula 142 to access the target vertebra 104 to create a hole and/or passageway in a bone of the target vertebra 104. In some cases, the awl and the trocar 134 may be introduced through the cannula 142 to access the target vertebra 104 to create passageways in soft tissues associated with the bone of the target vertebrae 104 and to withdraw fluid from the target vertebrae 104, respectively. Following the creation of the hole in the bone of the target vertebrae 104, the applicator device 136 may be employed to deliver the anchor implant 112 through the cannula 142 to the hole in the bone. Once the anchor implant 112 is anchored to the bone of the target vertebra 104, the line 102 that extends beyond the anchor implant 112 through the second calibrated hole 152 is carefully pulled by way of removal of the jig device 140 from the primary incision 154 such that the line 102 is extracted through the primary incision 154 while the jig device 140 is removed from the primary incision 154. In some embodiments of the present disclosure, the line 102 may be fastened using a bone anchor mechanism, to one of, but not limited to, adjacent vertebrae, to a previously placed spinal instrumentation (such as a plurality of spinal instrumentation 500 as shown in FIG. 5) via a clamp, a knot mechanism, to other areas of the spine, and the like to complete the minimally invasive tethering procedure.

FIG. 2 illustrates the drill bit 132, the trocar 134, and the applicator device 136, according to embodiments of the present disclosure. As discussed, prior to insertion of the anchor implant 112 into the cannula 110 of FIG. 1, the drill bit 132 may be inserted through the cannula 110, 142 (as shown in FIG. 1A and FIG. 1C) to create a hole in the target vertebra 104 (as shown in FIGS. 1A-1C). Specifically, the drill bit 132 may be attached to a guidewire or flexible cable 200 that allows passing the drill bit 132 through the cannula 110, 142 to access the bone of the target vertebra 104 as directed by the first calibrated hole 118, 150 and the second calibrated hole 124, 152 of the jig device 108, 140, respectively. In some embodiments of the present disclosure, when the drill bit 132 is used through a formal surgical exposure, the drill bit 132 is a straight drill bit that allows direct access to the guide 114 of the jig device 108 (as shown in FIG. 1A). In some embodiments of the present disclosure, when the drill bit 132 is used percutaneously, the drill bit 132 is a flexible and curved drill bit that allows for passage through the cannula 142 (i.e., the curved and flexible cannula) (as shown in FIG. 1C).

Similarly, to withdraw fluid from the target vertebra 104, the trocar 134 may be inserted through the cannula 110, 142 such that the trocar 134 is operated to withdraw fluid from the target vertebrae 104 prior to insertion of the anchor implant 112 into the cannula 110, 142. Specifically, the trocar 134 may be include a flexible tube 202 that allows passing the trocar 134 through the cannula 110, 142 to access the bone of the target vertebra 104 as directed by the first calibrated hole 118, 150 and the second calibrated hole 124, 152 of the jig device 108, 140 to withdraw fluid from the target vertebrae 104. In some embodiments of the present disclosure, when the trocar 134 is used through a formal surgical exposure, the trocar 134 is a straight trocar that allows direct access to the guide 114 of the jig device 108 (as shown in FIG. 1A). In some embodiments of the present disclosure, when the trocar 134 is used percutaneously, the trocar 134 includes a flexible and curved tube 202 that allows for passage through the curved cannula 142 (i.e., the curved and flexible cannula) (as shown in FIG. 1C).

Further, the applicator device 136 may be configured to facilitate insertion of the anchor implant 112 through the cannula 110, 142. Specifically, the applicator device 136 may be a specialized surgical instrument designed to facilitate the controlled delivery and precise placement of the anchor implant 112 through the cannula 110, 142 into the hole of the target vertebra 104. The applicator device 136 may include an elongated, rigid shaft sized to pass through the inner diameter of the cannula 110, 142, terminating in a coupling or engagement mechanism that interfaces with the proximal end of the anchor implant 112. This mechanism may utilize a threaded connection, snap-fit, friction grip, or other attachment means to maintain stable engagement during insertion. Additionally, the applicator device 136 may incorporate a handle or actuation mechanism at its proximal end, enabling the surgeon to apply controlled axial force or rotational motion to advance the anchor implant 112 into the target vertebra 104. Once the anchor implant 112 is properly positioned within the pre-formed bone cavity, the applicator device 136 may be disengaged and withdrawn, leaving the implant securely seated and connected to the line 102 for subsequent procedural steps. In some embodiments of the present disclosure, when the applicator device 136 is used through a formal surgical exposure, the applicator device 136 is a straight drill bit that allows direct access to the guide 114 of the jig device 108. In some embodiments of the present disclosure, when the applicator device 136 is used percutaneously, the applicator device 136 is a flexible and curved applicator device that allows for passage through the curved cannula 142 (i.e., the curved and flexible cannula) (as shown in FIG. 1C).

FIG. 3A illustrates an axial view 300 of the target vertebra 104 where the anchor implant 112 is installed onto a spinous process 302 of the target vertebra 104, according to an embodiment of the present disclosure. As illustrated, the system 100 having the jig device 108, 140 (as shown in FIG. 1A and FIG. 1C) and the cannula 110, 142 (as shown in FIG. 1A and FIG. 1C) may be appropriately positioned and the drill bit 132 (as shown in FIG. 2) may be inserted through the cannula 110, 142 to create a hole 304 in the spinous process 302 of the target vertebra 104. Once, the hole 304 is created, the anchor implant 112 may be delivered through the cannula 110, 142 to the hole 304 made in the spinous process 302. Specifically, the applicator device 136 (as shown in FIG. 2) may be used to pass the anchor implant 112 attached to the line 102 through the jig device 108, 140 and into the hole 304. In a preferred embodiment of the present disclosure, the anchor implant 112 may include a first tab 112a and a second tab 112b. As illustrated, the line 102 may be attached to the first tab 112a by wrapping the line 102 onto the tab 112a. Specifically, the first tab 112a may be coupled to a proximal end of the line 102. Further, the second tab 112b may have an opening (not shown) such that the second tab 112b is disposed over the line 102 via the opening formed therein. Such arrangement of the second tab 112b over the line 102 enables fixedly anchoring the anchor implant 112 to the bone (e.g., the spinous process 302). In some embodiments of the present disclosure, the first tab 112a may have an oblong shape. Specifically, due to the oblong shape, the first tab 112a has a width that allows a passage of the first tab 112a through the hole 304 in the spinous process 302 and/or associated soft tissue. Further, due to the oblong shape, the first tab 112a has a length that does not allow for a passage back through the same hole (i.e., the hole 304) when the first tab 112a is rotated in one of, a clockwise direction or an anticlockwise direction, of an axis on which the first tab 112a passes through the hole 304. In other words, due to the rotation of the first tab 112a in one of, the clockwise direction or the anticlockwise direction, of the axis on which the first tab 112a passes through the hole 304, the passage of the first tab 112a back through the hole 304 can be prevented by virtue of the oblong shape of the first tab 112a. Further, the second tab 112b that is disposed onto the line 102 may be configured to restrict a movement of the first tab 112a. Specifically, to restrict the movement of the first tab 112a, the second tab 112b may be pushed towards the first tab 112a such that the first tab 112a and the second tab 112b are held tightly on either side of the bone (e.g., the spinous process 302) of the target vertebra 104. In other words, the second tab 112b, which is disposed over the line 102, may be advanced towards the first tab 112a in a manner that causes the first tab 112a and the second tab 112b to securely clamp or sandwich the bone structure, such as the spinous process 302, therebetween. The positioning of the second tab 112b over the line 102 enables the second tab 112b to be translated along the line 102 toward the first tab 112a, thereby restricting relative movement between the first tab 112a and the second tab 112b. As a result, the bone (e.g., the spinous process 302) of the target vertebra 104 is securely engaged between the opposing tabs (i.e., the first tab 112a and the second tab 112b of the anchor implant 112), enhancing the stability and fixation of the anchor implant 112.

FIG. 3B illustrates another axial view 306 of the target vertebra 104 where the anchor implant 112 is installed onto the spinous process 302 of the target vertebra 104, according to an embodiment of the present disclosure. As illustrated, the system 100 having the jig device 108, 140 (as shown in FIG. 1A and FIG. 1C) and the cannula 110, 142 (as shown in FIG. 1A and FIG. 1C) may be appropriately positioned and the drill bit 132 (as shown in FIG. 2) may be inserted through the cannula 110, 142 to create the hole 304 in the spinous process 302 of the target vertebra 104. Once, the hole 304 is created, the anchor implant 112 may be delivered through the cannula 110, 142 to the hole 304 made in the spinous process 302. Specifically, the applicator device 136 may be used to pass the anchor implant 112 attached to the line 102 through the jig device 108, 140 and into the hole 304. In another embodiment of the present disclosure, the anchor implant 112 may include the first tab 112a and a second tab 308. As illustrated, the first tab 112a may have an opening (not shown) such that the proximal end 102a of the line 102 is inserted through the opening and fastened to the first tab 112a by way of, but not limited to, a knot, a permanent fixture, a bonding, adhesive, welding, a loop, and the like. Further, the second tab 308 may have an opening (not shown) such that the second tab 308 is disposed over the line 102 via the opening formed therein. Further, the second tab 308 may have a clamp device 310 that affixes the second tab 308 to the line 102 once advanced to a final position along the line 102. In other words, the second tab 308 may include a clamp device 310 that can be deployed to securely attach the second tab 308 to the line 102. Specifically, the clamp device 310 may be configured to engage when the second tab 308 is moved to an intended final position along the line 102, ensuring that the second tab 308 remains firmly fixed in place during use. Such arrangement of the second tab 308 over the line 102 enables fixedly anchoring the anchor implant 112 to the bone (e.g., the spinous process 302). As discussed, due to the oblong shape, the first tab 112a has the width that allows the passage of the first tab 112a through the hole 304 in the spinous process 302. Alternatively, the first tab may be passed through one or more holes made in associated soft tissue using an awl device. further, due to the oblong shape, the first tab 112a has the length that does not allow for a passage back through the same hole (i.e., the hole 304) when the first tab 112a is rotated in one of, the clockwise direction or the anticlockwise direction, of an axis on which the first tab 112a passes through the hole 304. Further, the second tab 308 may be pushed towards the first tab 112a and the second tab 308 may be secured by way of the clamp device 310 such that the first tab 112a and the second tab 308 are held tightly on either side of the bone (e.g., the spinous process 300) of the target vertebra 302 to enhance the stability and fixation of the anchor implant 112.

FIG. 3C illustrates another axial view 312 of the target vertebra 104 where the anchor implant 112 is installed onto the spinous process 302, according to an embodiment of the present disclosure. As illustrated, the system 100 having the jig device 108, 140 (as shown in FIG. 1A and FIG. 1C) and the cannula 110, 142 (as shown in FIG. 1A and FIG. 1C) may be appropriately positioned and the drill bit 132 (as shown in FIG. 2) may be inserted through the cannula 110, 142 to create the hole 304 in the spinous process 302 of the target vertebra 104. Once, the hole 304 is created, the anchor implant 112 may be delivered through the cannula 110, 142 to the hole 304 made in the spinous process 302. Specifically, the applicator device 136 may be used to pass the anchor implant 112 attached to the line 102 through the jig device 108, 140 and into the hole 304. In another embodiment of the present disclosure, the anchor implant 112 may include the first tab 112a and a second tab 314. As illustrated, the first tab 112a may be coupled to the proximal end 102a of the line 102. Further, the second tab 314 may have the opening (not shown) such that the second tab 314 is disposed over the line 102 via the opening formed therein. Further, the second tab 314 may include, but is not limited to, ridges, teeth, and the like, that permits sliding the second tab 314 over the line 102 in one direction. The one direction is towards the first tab 112a. Specifically, to restrict the movement of the first tab 112a, the second tab 314 may be pushed towards the first tab 112a such that the first tab 112a and the second tab 314 are held tightly on either side of the bone of the target vertebra 104 and the structure (e.g., the ridges and the teeth) disposed on the second tab 314 permits sliding the second tab 314 over the line 102 in only one direction towards the first tab 112a.

In each of the embodiments shown in FIGS. 3A-3C, in place of a hole being made in the spinous process, a hole may instead be made in soft tissue or hard tissue near or adjacent to the vertebrae. In such embodiments, the first tab 112a may inserted through the hole in the soft tissue or hard tissue and, after exiting the hole, rotated is a clockwise direction or anticlockwise direction, of an axis on which the first tab 112a passes through the hole.

FIG. 3D illustrates another axial view 316 of the target vertebra 104 where the anchor implant 112 is installed onto a bone of the target vertebra 104, according to an embodiment of the present disclosure. As illustrated, the system 100 having the jig device 108, 140 (as shown in FIG. 1A and FIG. 1C) and the cannula 110, 142 (as shown in FIG. 1A and FIG. 1C) may be appropriately positioned and the drill bit 132 (as shown in FIG. 2) may be inserted through the cannula 110, 142 to create the hole 304 in the spinous process 302 of the target vertebra 104. As illustrated, the hole 304 is a tunnel in the target vertebra 104 that enables anchoring of the anchor implant 112 in any bone other than the spinous process 302 (e.g., the lamina). Once, the hole 304 is created, the anchor implant 112 may be delivered through the cannula 110, 142 to the hole 304. Specifically, the applicator device 136 may be used to pass the anchor implant 112 attached to the line 102 through the jig device 108, 140 and into the hole 304. In an embodiment of the present disclosure, the anchor implant 112 has a first tab 112a that may have an opening (not shown) such that the proximal end of the line 102 is inserted through the opening and fastened to the first tab 112a by way of, but not limited to, a knot, a permanent fixture, a bonding, adhesive, welding, a loop, and the like. In some embodiments of the present disclosure, the first tab 112a may include a plurality of threads 318. Specifically, once the anchor implant 112 having the first tab 112a is received at the hole 304 through the cannula 110, the anchor implant 112 having the first tab 112a may be screwed inside the hole 304 to restrict the movement of the first tab 112a and thereby the anchor implant 112.

In some embodiments, for example, as shown in FIGS. 3E and 3F, the first tab 112 comprises a first end 340 and a second end 342 and is configured to fold with application of a force to each of the first end 340 and the second end 342 such that the first tab 112 may be inserted through the hole 304 of the target vertebra 104 in a folded position, such as, for example, through the spinous process 302, and the line 102 is coupled to the first tab 112, such that one end of the line 102 passes through the hole 304 together with the first tab 112. Upon passage of the folded first tab 112 through the hole 304 and removal of the forces on the first end 340 and the second end 342, the first tab 112 is configured to unfold. In an unfolded position, the first tab 112 is prevented from passing back through the hole 304. In some embodiments, the first tab 112 includes a spring 344 positioned between the first end 340 and the second end 342, such that upon application of a force to the first end 340 and the second end 342 a straightening force of the spring 344 is overcome resulting in the spring 344 being bent or flexed such that the first end 340 and the second end 342 are moved to a position adjacent to each other, shown in FIG. 3F. In certain embodiments, the spring 344 comprises a flat spring, a wound wire spring, a torsion spring, a compression spring, or any other type of spring. In some embodiments, the spring 344 may be comprises of metal, plastic, or some other biocompatible material

In some embodiments, the first tab 340, the second tab 342, or the spring 344 may include one or more antibacterial or antibiotic compounds.

FIGS. 4A-4C illustrate various views 400-404 of the target vertebra 104 showing holes and tunnels created for anchoring the anchor implant 112 and the line 102, according to embodiments of the present disclosure. FIG. 4A illustrates a coronal view 400 of the target vertebra 104. The figure depicts a plurality of holes 406 of which first through third holes 406a-406c are shown, created in the target vertebra 104. Each of the plurality of holes 406 may serve as an entry point for the anchor implant 112 and the associated line 102. FIG. 4B illustrates an axial view 402 of the target vertebra 104. The figure depicts a plurality of holes 408 of which first through fourth holes 408a-408d are shown, created in the target vertebra 104. Each of the plurality of holes 408 may serve as an entry point for the anchor implant 112 and the associated line 102 and allow for secure fixation within the target vertebra 104. FIG. 4C illustrates a sagittal view 404 of the target vertebra 104. The figure depicts a plurality of holes 410 of which first through fourth holes 410a-410d are shown, created in the target vertebra 104. Each of the plurality of holes 410 may serve as an entry point for the anchor implant 112 and the associated line 102.

FIG. 5 illustrates the system 100 installed on a coronal view of the vertebral column 101, according to embodiments of the present disclosure. The jig device 108 is inserted through the primary incision 126, carefully navigating beneath soft tissues to access the target vertebra 104. A secondary incision 128 allows for the insertion of the cannula 110 (as depicted in FIG. 1A), which passes through the calibrated holes 118, 124 (as depicted in FIG. 1A) of the jig device 108. The placement of the jig device 108 may be guided by various imaging techniques such as intraoperative X-ray, fluoroscopy, or 3D spinal navigation to ensure accurate localization. Once positioned, the cannula 110 is secured to the jig device 108 using the locking mechanism 130 (shown in FIG. 1B). Through the cannula 110, various instruments are introduced sequentially. Further, the applicator device 136 (as shown in FIG. 2) then delivers the anchor implant 112 with the attached line 102 through the cannula 110 and into the prepared vertebral hole. Once anchored, the guide 114 (as shown in FIG. 1A) is pulled out through the primary incision 126, extracting the line 102 along with it. The extracted line 102 can then be fastened to adjacent vertebrae, previously placed spinal instrumentation 500 of which first and second spinal instrumentations 500a and 500b are shown, or other areas of the spine using various mechanisms such as clamps or knots, completing the minimally invasive tethering procedure.

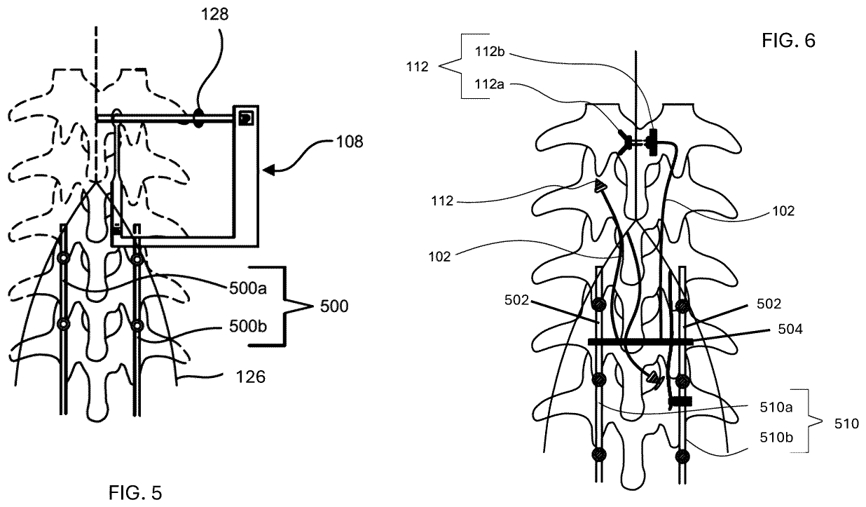

FIG. 6 illustrates a posterior view 600 of attachment of the line with the vertebral column 101 of the patient (as shown in FIG. 1), according to certain embodiments of the present disclosure. FIG. 6 shows how tether lines 102 are anchored to vertebrae using anchor implants 112. Each anchor implant 112 comprises a first tab 112a and a second tab 112b that work together to secure the tether line 102 to the vertebral structure.

The figure depicts vertebral holes 510, including a first vertebral hole 510a and a second vertebral hole 510b, through which the tether lines 102 are passed. These holes are created using the drill bit 132 introduced through the cannula 110 as described in the procedure. The tether lines 102 extend vertically along the spine, connecting multiple vertebral levels. The anchor implants 112 are positioned bilaterally on the vertebrae, creating a balanced configuration for the tethering system. The first tab 112a of each anchor implant 112 is passed through the respective vertebral hole 500, while the second tab 112b is positioned on the line 102 to restrict movement of the first tab 112a. The image demonstrates how the jig device 108, though not visible in this figure, has been used to guide the placement of the anchor implants 112 and tether lines 102. The lines 102 have been extracted through the primary incision and are now fastened to adjacent vertebrae or previously placed spinal instrumentation 500, including rods 502 and rod connectors 504. This configuration illustrates the versatility of the system, allowing for multi-level tethering and bilateral placement of anchor implants. It showcases the end result of the minimally invasive procedure, where the tether lines 102 are securely anchored to provide spinal stabilization or correction while minimizing tissue disruption.

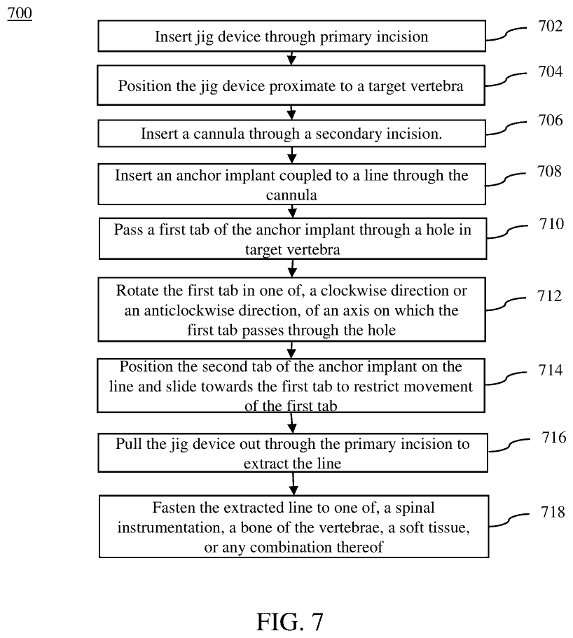

FIG. 7 illustrates a flowchart of a method 700 for anchoring the line 102 to the target vertebra 104, according to embodiments of the present disclosure.

At step 702, the jig device 108 may be inserted through the primary incision 126 made in the skin of the patient. The jig device 108 may include the guide 114 with the first calibrated hole 118 and the arm 116 with the second calibrated hole 124, as detailed in FIG. 1A.

Step 704 involves positioning the jig device 108 proximate to the target vertebra 104. This positioning may be guided by imaging techniques to ensure proper alignment with the intended vertebral structure.

At step 706, the cannula 110 may be inserted through the secondary incision 128. The cannula 110 may be carefully guided to pass through both the first calibrated hole 118 and the second calibrated hole 124 of the jig device 108, as shown in FIG. 1A and FIG. 1B.

Step 708 involves inserting the anchor implant 112 coupled to the line 102 through the cannula 110. This insertion may be facilitated by the applicator device 136, as illustrated in FIG. 2.

At step 710, the first tab 112a of the anchor implant 112 may be passed through the hole 304 formed in the target vertebra 104. Specifically, the hole 304 may have been previously created using the drill bit 132 passed through the cannula 110, as described in relation to FIG. 2 and FIG. 3A-3D.

Step 712 involves rotating the first tab 112a or expanding the first tab 112a once the first tab 112a has passed through the hole 304. The rotation may be in either a clockwise or anticlockwise direction around the axis on which the first tab 112a passes through the hole 304. The rotation takes advantage of the oblong shape of the first tab 112a, as described in FIG. 3A, to prevent it from passing back through the hole 304. If the first tab 112a includes a spring 344 as shown in FIGS. 3E and 3F, after passing through the hole 304, the first tab 112a expands under force of the spring 344.

At step 714, the second tab 112b of the anchor implant 112 may be positioned on the line 102 and slid towards the first tab 112a to restrict movement of the first tab 112a. This step may involve different configurations of the second tab, such as the clamp device 310 shown in FIG. 3B or the ratcheting mechanism of the tab 314 in FIG. 3C.

Step 716 involves pulling the jig device 108 out through the primary incision 126, thereby extracting the line 102. Specifically, the extraction brings the line 102 out of the patient's body for further manipulation.

Finally, at step 718, the extracted line 102 may be fastened to one or a combination of spinal instrumentation (such as the spinal instrumentations 500), vertebral bone, or soft tissue. This fastening may involve the use of additional hardware not explicitly shown in the figures but included in the system 100.

The method 700 may be repeated for multiple vertebrae, allowing for the creation of a multi-level tethering system as depicted in FIG. 6, where multiple anchor implants 112 and lines 102 are shown securing various levels of the spine.

Thus, the system 100, 138 and the associated methods (e.g., the method 700) provide several technical advantages. The minimally invasive approach allows for anchorage of lines to vertebrae with reduced soft tissue dissection, preserving the protective effects of natural soft tissues on spinal stability. The jig device 108, 140 enables precise targeting and alignment for instrument passage and implant placement, enhancing procedural accuracy. The flexible, curved instruments (e.g., cannula 142, drill bit 132, trocar 134, and applicator device 136) facilitate access to vertebral structures through small incisions, reducing surgical trauma. The anchor implant 112 with its tab design (e.g., first tab 112a and second tab 112b, 308, 314) provides secure fixation through or within vertebral bone while allowing for controlled tensioning of the line 102. The system's versatility accommodates both formal surgical exposures and percutaneous applications, adapting to various clinical scenarios. Additionally, the method allows for connection of the line 102 to existing spinal instrumentation, enhancing its utility in complex spinal procedures.

Although preferred embodiments have been depicted and described in detail herein, it will be apparent to those skilled in the relevant art that various modifications, additions, substitutions and the like can be made without departing from the spirit of the disclosure, and these are, therefore, considered to be within the scope of the disclosure, as defined in the following claims. Features or functionality described with respect to certain example embodiments may be combined and sub-combined in and/or with various other example embodiments. Also, different embodiments and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

The terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc. can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from the teachings of the present disclosure.

The terminology used herein is for describing particular example embodiments and is not intended to be necessarily limiting of the present disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence and/or addition of one or more other features, integers, steps, operations, elements, components, and/or groups thereof.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

As used herein, the term “about” and/or “substantially” refers to a +/−10% variation from the nominal value/term. Such variation is always included in any given.

If any disclosures are incorporated herein by reference and such disclosures conflict in part and/or in whole with the present disclosure, then to the extent of conflict, and/or broader disclosure, and/or broader definition of terms, the present disclosure controls. If such disclosures conflict in part and/or in whole with one another, then to the extent of conflict, the later-dated disclosure controls.

The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Whereas many alterations and modifications of the present invention will no doubt become apparent to a person of ordinary skill in the art after having read the foregoing description, it is to be understood that the particular embodiments shown and described by way of illustration are in no way intended to be considered limiting.

1. A method for anchoring a line to a first vertebra, the method comprising:
inserting a guide of a jig device through a primary incision made in a skin of a patient, wherein the guide has a first calibrated hole;
positioning an arm of the jig device proximate to the first vertebra, wherein the arm is coupled to the guide and has a second calibrated hole;
inserting a cannula through a secondary incision made in the skin of the patient while the cannula passes through the first calibrated hole and the second calibrated hole;
inserting an anchor implant coupled to the line through the cannula, wherein the anchor implant comprising a first tab and a second tab;
passing the first tab through a hole in the first vertebra to anchor the anchor implant at a distal side of the first vertebra;
positioning the second tab on the line on a proximal side of the first vertebra to restrict movement of the first tab;
pulling the guide out through the primary incision, thereby extracting the line through the primary incision; and
fastening the extracted line to at least one of, a spinal instrumentation, a bone of a second vertebra, a soft tissue, or a combination thereof.

2. The method of claim 1, comprising rotating the first tab once the first tab exits the hole on the distal side of the first vertebra in one of, a clockwise direction or an anticlockwise direction, to prevent passage back of the first tab through the hole once inserted by virtue of an oblong shape of the first tab.

3. The method of claim 1, further comprising:
locking the cannula to the jig device using a locking mechanism once the cannula is inserted into the first calibrated hole and the second calibrated hole.

4. The method of claim 1, wherein prior to inserting the anchor implant, the method comprising:
inserting a drill bit through the cannula to create the hole in the first vertebra;
inserting an awl through the cannula to create passageways in soft tissues associated with the first vertebra; or
inserting a trocar through the cannula to withdraw fluid from the first vertebra.

5. The method of claim 1, wherein positioning the second tab comprising:
deploying a clamp device on the second tab to affix the second tab to the line once advanced to a final position along the line on the proximal side of the first vertebra.

6. The method of claim 1, wherein positioning the second tab comprising:
sliding the second tab over the line in one direction towards the first tab, wherein the second tab comprises one of, ridges or teeth, that permits sliding in only the one direction.

7. A method for anchoring a line to a first vertebra, the method comprising:
inserting a jig device having first and second calibrated holes through a primary incision made in a skin of a patient;
inserting a curved cannula through a secondary incision made in the skin of the patient such that the curved cannula passes through the first calibrated hole and the second calibrated hole;
inserting an anchor implant coupled to the line through the curved cannula, wherein the anchor implant comprising a first tab and a second tab;
passing the first tab through a hole in the first vertebra to anchor the anchor implant at a distal side of the first vertebra;
positioning the second tab on the line on a proximal side of the first vertebra to restrict movement of the first tab;
pulling the guide out through the primary incision, thereby extracting the line through the primary incision; and
fastening, by way of a fastener, the extracted line to at least one of, a spinal instrumentation, a bone of a second vertebra, a soft tissue, or a combination thereof.

8. The method of claim 7, comprising rotating the first tab once the first tab exits the hole on the distal side of the first vertebra in one of, a clockwise direction or an anticlockwise direction, to prevent passage back of the first tab through the hole once inserted by virtue of an oblong shape of the first tab.

9. The method of claim 7, further comprising:
locking the cannula to the jig device using a locking mechanism once the cannula is inserted into the first calibrated hole and the second calibrated hole.

10. The method of claim 7, wherein positioning the second tab comprising:
deploying a clamp device on the second tab to affix the second tab to the line once advanced to a final position along the line on the proximal side of the first vertebra.

11. The method of claim 7, wherein positioning the second tab comprising:
sliding the second tab over the line in one direction towards the first tab, wherein the second tab comprises one of, ridges or teeth, that permits sliding in only the one direction.

12. A system comprising:
a jig device comprising:
a guide having a first calibrated hole, wherein the guide is configured to be inserted through a primary incision made in a skin of a patient; and
an arm that is coupled to the guide, and has a second calibrated hole;
a cannula configured to be inserted through a secondary incision made in the skin of the patient such that the cannula passes through the first calibrated hole and the second calibrated hole;
an anchor implant that is coupled to a line, and configured to be inserted through the cannula, wherein the anchor implant comprising:
a first tab coupled to a proximal end of the line such that the first tab passes through a hole in a first vertebra to anchor the anchor implant at a distal side of the first vertebra; and
a second tab that is disposed onto the line on a proximal side of the first vertebra, and configured to restrict a movement of the first tab,
wherein, once the anchor implant is anchored at the distal side of the first vertebra, the guide is configured to be pulled out through the primary incision thereby extracting the line through the primary incision such that the line is fastened to one of one or more spinal instrumentations, a bone of a second vertebra, or a combination thereof.

13. The system of claim 12, wherein the guide has a first end and a second end such that the first end of the guide comprising the first calibrated hole.

14. The system of claim 13, wherein the arm is an L-shaped structure having a first end and a second end such that first end of the arm comprising the second calibrated hole and the second end of the arm is coupled to the second end of the guide.

15. The system of claim 12, wherein the first tab has an oblong shape such that when the first tab is passed through the hole to the distal side of the first vertebra, the first tab is rotated by way of the line in one of a clockwise direction or an anticlockwise direction, to prevent passage back of the first tab through the hole to the proximal side once inserted by virtue of the oblong shape.

16. The system of claim 12, further comprising a locking mechanism configured to lock the cannula to the jig device once the cannula is inserted into the first calibrated hole and the second calibrated hole.

17. The system of claim 12, further comprising a drill bit, wherein, prior to insertion of the anchor implant into the cannula, the drill bit is configured to be inserted through the cannula to create a hole in the first vertebra.

18. The system of claim 12, further comprising an awl, wherein, prior to insertion of the anchor implant into the cannula, the awl is configured to be inserted through the cannula to create passageways in soft tissues associated with the bone of the first vertebra.

19. The system of claim 12, further comprising a trocar, wherein, prior to insertion of the anchor implant into the cannula, the trocar is configured to be inserted through the cannula to withdraw fluid from the first vertebra.

20. The system of claim 12, further comprising a fastener configured to fasten the line that is extracted from the primary incision with one of one or more spinal instrumentations, a bone of a second vertebrae, a soft tissue, or a combination thereof.

21. The system of claim 12, wherein the second tab comprising a deployable clamp device that affixes the second tab to the line once advanced to a final position along the line on the proximal side of the first vertebra.

22. The system of claim 12, wherein the second tab comprising one of, ridges or teeth, that permits sliding the second tab over the line in one direction, wherein the one direction is towards the first tab.

23. A system comprising:
a jig device that is configured to be inserted through a primary incision made in a skin of a patient, wherein the jig device comprising a first calibrated hole and a second calibrated hole;
a curved cannula configured to be inserted through a secondary incision made in the skin of the patient such that the curved cannula passes through the first calibrated hole and the second calibrated hole;
an anchor implant that is attached to a line, and configured to be inserted through the curved cannula, wherein the anchor implant comprising:
a first tab attached to a proximal end of the line such that the first tab passes through a hole of a bone of a first vertebra to anchor the anchor implant at a distal side of the bone; and
a second tab that is disposed on the line at a proximal side of the bone, and configured to restrict a movement of the first tab,
wherein, once the anchor implant is anchored at the distal side of the bone, the guide is configured to be pulled out through the primary incision thereby extracting the line through the primary incision such that the line is fastened to one of one or more spinal instrumentations, a bone of a second vertebrae, or a combination thereof.

24. The system of claim 23, wherein the jig device has a L-shaped structure, wherein the jig device comprising a first end and a second end such that the first end of the jig device comprising the first calibrated hole and the second end of the jig device comprising the second calibrated hole.

25. The system of claim 23, wherein the first tab has an oblong shape, wherein when the first tab is passed through the hole at the distal side of the first vertebra, the first tab is rotated in one of, a clockwise direction or an anticlockwise direction, to prevent passage back through the hole to the proximal side by virtue of the oblong shape.

26. The system of claim 23, further comprising a locking mechanism configured to lock the cannula to the jig device once the cannula is inserted into the first calibrated hole and the second calibrated hole.

27. The system of claim 23, further comprising:
a curved and flexible drill bit, wherein, prior to insertion of the anchor implant into the curved cannula, the curved and flexible drill bit drill bit is inserted through the curved cannula to create a hole in the bone of the vertebrae;
a curved and flexible awl, wherein, prior to insertion of the anchor implant into the curved cannula, the curved and flexible awl is inserted through the curved cannula to create passageways in soft tissues associated with the bone of the vertebrae; and
a curved and flexible trocar, wherein, prior to insertion of the anchor implant into the curved cannula, the curved and flexible trocar is inserted through the curved cannula to withdraw fluid from the target vertebrae.

28. A system comprising:
a jig device that is configured to be inserted through a primary incision made in skin of a patient, wherein the jig device comprising a first calibrated hole and a second calibrated hole;
a cannula configured to be inserted through a secondary incision made in the skin of the patient such that the cannula passes through the first and second calibrated holes;
an anchor implant that is attached to a line, and configured to be inserted through the curved cannula, wherein the anchor implant is comprising a plurality of threads such that the anchor implant is screwed inside the hole,
wherein, once the anchor implant is screwed inside the hole of the bone, the jig device is pulled out through the primary incision thereby extracting the line through the primary incision such that the line is fastened to one of, one or more spinal instrumentations, a bone of the vertebrae, or a combination thereof.

29. The system of claim 28, wherein the jig device has a L-shaped structure, wherein the jig device comprising a first end and a second end such that the first end of the jig device comprising the first calibrated hole and the second end of the jig device comprising the second calibrated hole.

30. The system of claim 28, wherein the jig device comprising:
a guide having a first calibrated hole, wherein the guide is configured to be inserted through the primary incision made in the skin of the patient; and
an arm that is coupled to the guide, and has the second calibrated hole.

31. The system of claim 30, wherein the guide is pulled out through the primary incision thereby extracting the line through the primary incision.

32. The system of claim 28, wherein the cannula is a straight cannula.

33. The system of claim 28, wherein the cannula is a curved cannula.

34. The system of claim 28, wherein the first tab has an oblong shape, wherein when the first tab is passed through the hole of the target vertebra, the first tab is rotated in one of, a clockwise direction or an anticlockwise direction, to prevent passage back through the hole by virtue of the oblong shape.

35. The system of claim 28, further comprising a locking mechanism configured to lock the cannula to the jig device once the cannula is inserted into the first and second calibrated holes.

36. The system of claim 28, further comprising:
a drill bit, wherein, prior to insertion of the anchor implant into the cannula, the curved and flexible drill bit drill bit is configured to be inserted through the cannula to create a hole in the bone of the vertebrae;
an awl, wherein, prior to insertion of the anchor implant into the cannula, the awl is configured to be inserted through the cannula to create passageways in soft tissues associated with the bone of the vertebrae; and
a trocar, wherein, prior to insertion of the anchor implant into the cannula, the trocar is configured to be inserted through the cannula to withdraw fluid from the target vertebrae.

37. The system of claim 36, wherein:
the drill bit is a curved and flexible drill bit;
an awl is a curved and flexible awl; and
the trocar is a curved and flexible trocar.

38. A system comprising:
a jig device configured to be inserted through a primary incision made in skin of a patient, wherein the jig device comprising first and second calibrated holes:
a cannula configured to be inserted through a secondary incision made in the skin of the patient such that the cannula passes through the first and second calibrated holes;
an anchor implant that is coupled to a line, and configured to be inserted through the cannula, wherein the anchor implant is configured to be anchored to a hole in the target vertebra;
wherein the jig device configured to be pulled out through the primary incision when the anchor implant is anchored to the target vertebra thereby extracting the line through the primary incision such that the line is fastened to one of, one or more spinal instrumentations, a bone of the vertebrae, or a combination thereof.

39. The system of claim 38, wherein the jig device comprising:
a guide having a first end and a second end such that the first end of the guide comprising the first calibrated hole; and
an arm having a first end and a second end such that first end of the arm comprising the second calibrated hole and the second end of the arm is coupled to the second end of the guide.

40. The system of claim 38, wherein when the jig device has the guide and the arm, the cannula used is a straight cannula.

41. The system of claim 38, the cannula is a curved and flexible cannula.

42. The system of claim 38, wherein the anchor implant comprising:
a first tab coupled to a proximal end of the line such that the first tab passes through the hole in the target vertebra to anchor the anchor implant to the target vertebra, wherein the first tab has an oblong shape such that when the first tab is passed through the hole of the target vertebra, the first tab is rotated by way of the line in one of, a clockwise direction or an anticlockwise direction, to prevent passage back of the first tab through the hole once inserted by virtue of the oblong shape; and
a second tab that is disposed onto the line, and configured to restrict a movement of the first tab.

43. The system of claim 38, wherein the anchor implant comprising a first tab having a plurality of threads such that when the first tab of the anchor implant is received at the hole through the cannula, the first tab is screwed inside the hole to anchor the anchor implant.

44. The system of claim 38, further comprising a locking mechanism configured to lock the cannula to the jig device once the cannula is inserted into the first and second calibrated holes.

45. The system of claim 38, further comprising:
a drill bit, wherein, prior to insertion of the anchor implant into the cannula, the drill bit is configured to be inserted through the cannula to create a hole in the target vertebra.
an awl, wherein, prior to insertion of the anchor implant into the cannula, the awl is configured to be inserted through the cannula to create passageways in soft tissues associated with the bone of the vertebrae; and
a trocar, wherein, prior to insertion of the anchor implant into the cannula, the trocar is configured to be inserted through the cannula to withdraw fluid from the target vertebrae.

46. The system of claim 38, wherein when the jig device has the guide and the arm:
the drill bit used is a straight drill bit;
an awl used is a straight awl; and
the trocar used is a straight trocar.

47. The system of claim 38, wherein the second tab comprising a clamp device that affixes the second tab to the line once advanced to a final position along the line.

48. The system of claim 38, wherein the second tab comprising one of, ridges or teeth, that permits sliding the second tab over the line in one direction, wherein the one direction is towards the first tab.

49. An anchor implant for a vertebra, the anchor implant comprising:
a first tab attached to a proximal end of a line such that the first tab passes through a hole of a bone of a first vertebra to anchor the anchor implant at a proximal side of the bone, wherein the first tab has an oblong shape such that when the first tab exits through the hole of the bone at the distal side, the first tab is rotated in one of, a clockwise direction or an anticlockwise direction, to prevent passage back through the hole to the proximal side by virtue of the oblong shape of the first tab; and
a second tab that is disposed on the line on the proximal side, and configured to restrict a movement of the first tab, wherein to restrict the movement of the first tab, the second tab is pushed towards the first tab such that the first tab and the second tab are held tightly on either side of the bone of the first vertebra.

50. The anchor implant of claim 49, wherein the first tab and the second tab attached to the line are passed through a cannula coupled to a jig device having a first calibrated hole and a second calibrated hole when the jig device is positioned proximate to the first vertebra.
